(19) United States
(12) Patent Application Publication
(10) Pub. No.: US 2025/0318982 A1
(43) Pub. Date: Oct. 16, 2025

(54) APPLICATOR HAVING A MASSAGE HEAD FOR DISPENSING A LIQUID

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Feng Jianxin, Shenzhen (CN); Guo Chunfang, Shenzhen (CN)

(73) Assignee: SHENZHEN NUON MEDICAL EQUIPMENT CO., LTD., Shenzhen (CN)

(21) Appl. No.: 18/822,547

(22) Filed: Sep. 3, 2024

(30) Foreign Application Priority Data
Apr. 16, 2024 (CN) 2024104579587
Apr. 16, 2024 (CN) 2024207891580
Apr. 16, 2024 (CN) 2024208083660
Jun. 4, 2024 (CN) 2024212613509

(51) Int. Cl. A61H 15/02; A45D 34/00; A45D 34/04; A61H 15/00; A61N 5/06

(52) U.S. Cl. CPC A61H 15/02; A45D 34/04; A61H 15/0092; A61N 5/0616; A45D 2034/002; A45D 2200/205; A45D 2200/207; A61H 2015/0028; A61H 2201/10; A61H 2201/105; A61N 2005/0644; A61N 2005/0652; A61N 2005/0663

(57) ABSTRACT

Embodiments of the present invention provide an apparatus for applying a liquid from a bottle and a massage head. The apparatus comprises a housing having a liquid inlet end configured to be connected to a liquid storage bottle, a first liquid discharge channel in the housing and a liquid discharge head body having a second liquid discharge channel. The first liquid discharge channel is connected to the second discharge channel through a drain pipe. An elastic piece is positioned between the housing and the liquid discharge head body. The elastic piece compress or decompress under an action of an external force. The elastic piece allows 360 degree rotation to the applicator and applicator bends at a predetermined angle.

### TECHNICAL FIELD

The present invention relates generally to an apparatus for dispensing a liquid. More specifically, the present invention relates to an applicator for applying cosmetics.

### BACKGROUND ART

In order to improve the uniformity of skin care cosmetics used on the face, makeup products are usually applied to the face with the help of a massage head. Specifically, the cosmetic product is installed on the massage head, and the liquid in the cosmetic product can be discharged from the liquid outlet end of the massage head to apply the liquid evenly.

However, the liquid outlet end of the current massage head is adapted to the skin by adopting an inclined plane or arc structure, and the massage head cannot adjust the application angle according to the shape of the face, which leads to the poor application effect of the massage head.

In conventional devices, the massage head is usually used as an auxiliary device, specifically the cosmetic product is installed on the massage head, and the liquid in the cosmetic product can be discharged from the liquid outlet end of the massage head to achieve the purpose of uniform application.

In order to incorporate phototherapy with makeup products, a light therapy lamp plate is usually installed in the massage head, which can be used to provide phototherapy on the face's skin to improve the face's treatment effect. However, the phototherapy lamp panel is usually bonded and fixed on the inner wall of the massage head, which makes the installation and positioning of the phototherapy lamp panel complicated, complex to disassemble, and inconvenient for maintenance.

### OBJECTS OF THE INVENTION

Some of the objects of the invention are as follows:

An object of the present invention is to provide an applicator with an outlet end rotatable around axis and can be bent at an angle to adjust according to the face contour of a user.

Another object of the present invention is to provide an applicator with a better application effect of the massage head.

Another object of the present invention is to provide an applicator with a massage head where the installation and positioning of the board containing the stimulation element is convenient and disassembly is easy.

### SUMMARY OF THE INVENTION

According to a first aspect of the present invention, an applicator for applying a liquid is provided. The applicator comprising: a housing having a liquid inlet end configured to be connected to a liquid storage bottle; a first liquid discharge channel in the housing; a liquid discharge head body having a second liquid discharge channel; a drain pipe to connect the first liquid discharge channel with the second liquid discharge channel; and an elastic piece positioned between the housing and the liquid discharge head body, the elastic piece compress or decompress under an action of an external force.

In one embodiment of the invention, the elastic piece compresses when the liquid discharge head body swings around the housing, under the action of the external force and decompresses when the external force is removed.

In one embodiment of the invention, the elastic piece allows 360 degree rotation of the liquid discharge head body and tilting the liquid discharge head body at an angle.

In one embodiment of the invention, an inner side wall of the housing extends inward to form a resisting seat, the resisting seat is positioned between a first step portion of the liquid discharge head body and a second step portion of the elastic piece.

In one embodiment of the invention, a first end of the liquid discharge head body has an insert plate that inserts into a slot provided on the elastic piece.

In one embodiment of the invention, the elastic piece is provided with a refuge to allow the first liquid discharge channel to pass through, and the inner side wall of the refuge is convex inward and is provided with boss.

In one embodiment of the invention, the housing further comprises a bracket for supporting the elastic piece.

In one embodiment of the invention, the applicator further comprising a conductive needle installed on the liquid discharge head body and a charging unit for charging a battery.

According to a second aspect of the present invention, an applicator for applying a liquid is provided. The applicator comprising: a housing having a liquid inlet end configured to be connected to a liquid storage bottle; a first liquid discharge channel in the housing; a liquid discharge head body having a second liquid discharge channel connected to the first liquid discharge channel; wherein the liquid discharge head body comprising: a massage head body having a first through hole formed in center of the massage head body; a first plate having at least one stimulation element arranged in the massage head body, the first plate has a second through hole in center in a position corresponding to the first through hole; a connecting guide rod that passes through the first through hole and the second through hole; wherein the connecting guide rod has a liquid outlet hole.

In one embodiment of the invention, the applicator further comprises a locking piece to clamp the first plate with the inner side of the massage head body, the locking piece detachably connects with the connecting guide rod above the second through hole of the first plate.

In one embodiment of the invention, the connecting guide rod has an annular guide groove and the locking piece comprises a snap ring that snuggly fits into the annular guide groove on the connecting guide rod.

In one embodiment of the invention, the at least one stimulation element is selected from a group consisting of Light Emitting Diodes (LEDs), lasers, heating elements, cooling elements, vibration elements, electrodes, micro-current element and combinations thereof.

In one embodiment of the invention, the massage head comprises a partition plate and the first through hole is at the center of the partition plate.

In one embodiment of the invention, the partition plate comprises a light transmission hole corresponding to the position of the at least one stimulation element on the first plate.

In one embodiment of the invention, the massage head further comprises a lens positioned above the partition plate, the lens has a third through hole for the connecting guide rod to pass through.

In one embodiment of the invention, the partition plate comprises one or more positioning grooves, and the first plate comprises one or more positioning piece to fit into the one or more positioning grooves on the first plate.

In one embodiment of the present invention, the connecting guide rod comprises a liquid outlet hole.

In one embodiment of the present invention, the first plate comprises one or more avoidance notch arranged at intervals with the second through hole, and an inner side wall of the massage head body is provided with one or more positioning guide rod for inserting into the one or more avoidance notch.

In one embodiment of the present invention, the applicator further comprising a top portion connected to the massage head body, the top portion has a housing for accommodating a main circuit board and a battery.

According to a third aspect of the present invention, an applicator for applying a liquid is provided. The applicator comprising: a housing having a liquid inlet end configured to be connected to a liquid storage bottle; a first liquid discharge channel in the housing; a liquid discharge head body having a second liquid discharge channel; a drain pipe to connect the first drain channel with the second liquid discharge channel; and wherein the liquid discharge head body is transparent and includes a luminescent filament in a hose that emits a color light.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first’, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a connection between an applicator and a liquid reservoir in accordance with an embodiment of the present invention.

FIG. 2 is an exploded view of the applicator in accordance with an embodiment of the present invention.

FIG. 3 is a cross-sectional representation of an applicator in accordance with an embodiment of the present invention.

FIG. 4 is an exploded view of a shell of the applicator in accordance with an embodiment of the present invention.

FIG. 5 is a cross-sectional representation of the shell in accordance with an embodiment of the present invention.

FIG. 6 illustrates a structure of a bottom shell in accordance with an embodiment of the present invention.

FIG. 7 is an exploded view of the discharge head body in accordance with an embodiment of the present invention.

FIG. 8 is a cross-sectional representation of the discharge head body in accordance with an embodiment of the present invention.

FIG. 9 illustrates a plate containing stimulation element in accordance with an embodiment of the present invention.

FIG. 10 illustrates a base of the massage head in accordance with an embodiment of the present invention.

FIG. 11 illustrates an elastic piece configuration in accordance with an embodiment of the present invention.

FIG. 12 is a cross-sectional representation of a charging unit in accordance with an embodiment of the present invention.

FIG. 13 is an exploded view showing a cover of the applicator in accordance with an embodiment of the present invention.

FIG. 14 illustrates an inner tube in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose an applicator for applying a liquid from a bottle body. The apparatus comprises a shell having a liquid inlet end configured to be connected to a liquid storage bottle, a first liquid discharge channel in the shell and a liquid discharge head body having a second liquid discharge channel. The first liquid discharge channel is connected to the second discharge channel through a drain pipe. An elastic piece is positioned between the shell and the liquid discharge head body. The elastic piece compress or decompress under an action of an external force. The elasti piece allows 360 degree rotation to the applicator and applicator bends at a predetermined angle.

Another embodiment of the present invention discloses an applicator for applying a liquid and a massage head for providing massage to a user's skin. The massage head comprises a massage head body having a first through hole formed in centre of the massage head body, a first plate having at least one stimulation element arranged in the massage head body, the first plate has a second through hole in centre in a position corresponding to the first through hole, a connecting guide rod that passes through the first through hole and the second through hole, and a locking piece to clamp the first plate with an inner side of the massage head body, the locking piece detachably connects with the connecting guide rod above the second through hole of the first plate.

Several additional functionalities may also be added to the apparatus and the cleaning device. For example, the apparatus may have one or more sensors to sense the pH and monitor one or more parameters of the skin of a user. Moreover, activation and control of specific stimulation elements may be based upon feedback received from the one or more sensors, such as pressure sensors (for ascertaining contact with a body portion of the user) and temperature sensors (for maintaining the temperature of heating and/or cooling elements within permissible limits) about several parameters, such as skin temperature, treatment time, skin pH. Electrical power to operate the device may be provided through an onboard rechargeable battery that may be charged using a power cable. The apparatus can also be controlled by a user computing device communicating with the apparatus over a communication network through a communication interface built into the therapeutic device.

Several embodiments of the present invention will now be described in detail with references to FIGS.

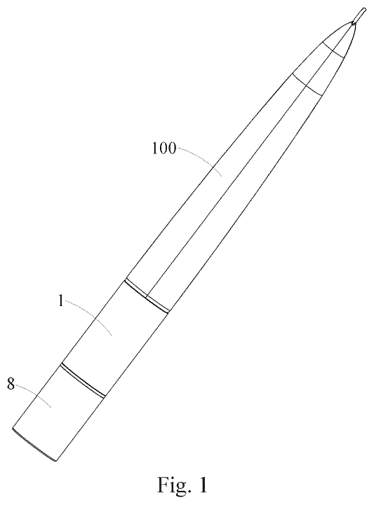

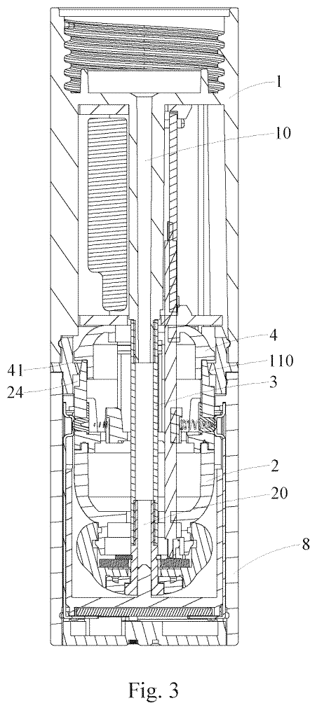

Referring to FIG. 1 to FIG. 3, an applicator is described. This applicator comprises a housing 1, a liquid discharge head body 2, a drain pipe 3 and an elastic piece 4. The housing 1 contains a first liquid discharge channel 10 and the two ends of the housing 1 are a liquid inlet end and a liquid outlet end. The liquid inlet end and the liquid outlet end of the housing 1 are the two ends of the first liquid discharge channel 10. The inlet end of housing 1 is used for connection with a bottle or a reservoir 100 containing a liquid. The bottle or reservoir 100 can be a cosmetic products, such as eye cream, etc. The liquid discharge head body 2 contains a second liquid discharge channel 20. The two ends of the liquid discharge head body 2 are a liquid inlet end and a liquid outlet end, and the liquid inlet end and the liquid outlet end of the liquid discharge head body 2 are the two ends of the second liquid discharge channel 20. A drain pipe 3 communicates the first liquid discharge channel 10 with the second liquid discharge channel 20. The drain pipe 3 communicates the liquid outlet end of the first liquid discharge channel 10 with the liquid inlet end of the second liquid discharge channel 20. The elastic piece 4 connects the liquid outlet end of housing 1 and the liquid inlet end of liquid discharge head body 2. The elastic piece 4 can be a silicone ring or a spring, etc. In a preferred embodiment, the elastic piece 4 is a silica gel ring. In the process of contact between the liquid discharge head body 2 and the face, the elastic piece 4 can be tilted and rotated telescopically by the action of external force, such that the liquid discharge head body 2 can swing around the housing 1, specifically reciprocating motion in the direction of the axis of the first liquid discharge channel 10. When the external force is removed, the elastic piece 4 pushes the discharge head body 2 to reset (as shown in FIG. 3). The first liquid discharge channel 10 is in communication with the second liquid discharge channel 20 through the liquid drain pipe 3, and the liquid in the bottle or reservoir 100 connected with the housing 1 can pass through the first liquid discharge channel 10 to the drain pipe 3 and then to the second liquid discharge channel 20. The liquid is discharged by the liquid outlet end of the liquid discharge head body 2 to realize coating. The liquid outlet end of the housing 1 is connected with the liquid inlet end of the liquid discharge head body 2 through the elastic piece 4. The liquid discharge head body 2 extrudes the elastic piece 4 and compresses and deforms in the process of contact with the face. The liquid discharge head body 2 can swing around the housing 1 due to the elastic piece 4, such that the position of the liquid discharge head body 2 can be adjusted depending on the movement of the applicator over the facial contour of the user, so that the coating angel can be adjusted and coating can be applied uniformly thereby improving the coating effect.

The first liquid discharge channel 10, the drain pipe 3 and the second liquid discharge channel 20 are arranged coaxially.

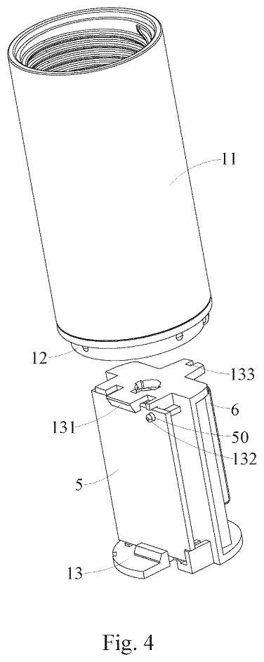

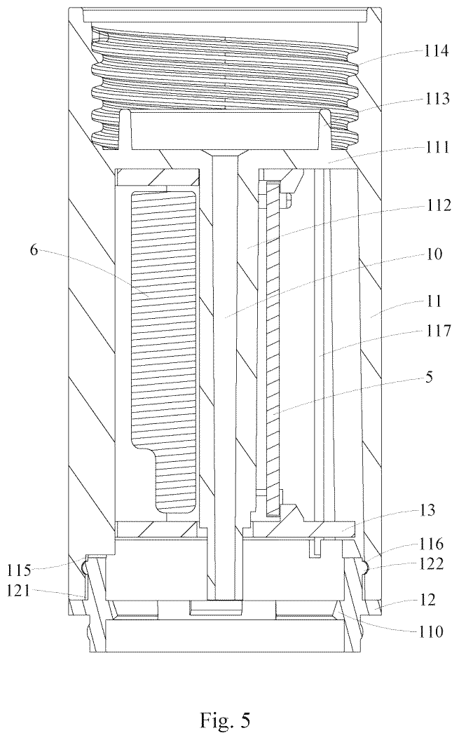

Referring to FIG. 4 and FIG. 5, the housing 1 comprise a shell 11, a bottom shell 12 installed at the outlet end of the shell 11 and a bracket 13 installed in the shell 11. The first liquid discharge channel 10 is provided inside the shell 11, and the first liquid discharge channel 10 penetrates through the bracket. A first end of the elastic piece 4, is connected with the bracket 13. The inner side wall of the bottom shell 12 is provided with a resisting seat 110. The configuration of the shell 11 is roughly cylindrical, and the bottom shell 12 is also cylindrical, corresponding to shell 11. The inner side wall of the shell 11 extends inward with an extension plate 111. The extension plate 111 extends along the axis direction of the shell 11 to form a guide pillar 112. The extension guide pillar penetrates through the bracket 13 and the first liquid discharge channel is present in the guide pillar 112. The extension plate 111 extends away from the direction of the guide pillar 112 to form an annular side plate 113. The shell 11 is provided with an internal thread 114 on the inner side wall of the liquid inlet end, so that it can be threaded with the external thread of the bottle or reservoir 100 to realize the detachable connection between the bottle or reservoir 100 and the housing 1.

Referring to FIG. 5, the shell 11 is provided with a first step 115 on the inner side wall of the liquid outlet end, and the outer side wall of the bottom shell 12 is provided with a second step 121 that coordinates with the first step 115, so as to position the bottom shell 12 with the shell 11.

Referring to FIG. 5, a positioning groove 116 is provided in the first step 115. The second step 121 on the bottom shell 12 is provided with a positioning clamp 122 that inserts into the positioning groove 116. The connection stability between the bottom shell 12 and the shell 11 is improved by positioning and matching of the positioning clamp 122 and the positioning groove 116. The positioning groove 116 and the positioning clamp 122 are in annular structure, so that the connection strength between the bottom shell 12 and the shell 11 can be increased.

Referring to FIG. 3 and FIG. 5, the inner side wall of housing 1 extends inward to form a resisting seat 110. The discharge head body 2 is provided with a first step portion 24 and the elastic piece 4 is provided with a second step portion 41. The resisting seat 110 is arranged between the first step portion 24 and the second step portion 41. Specifically, the resisting seat 110 can be arranged on the inner side wall of the bottom shell 12. The first step portion 24 may be arranged at a first end of the base 21 close to the elastic piece 4, and the first end of the base 21 can be extended into the second step portion 41 to realize positioning, so that the force of the base 21 is conducive to making the base 21 evenly balanced, so that the liquid discharge head body 2 can remain erect after resetting. In this structure, when the liquid discharge head body 2 is in contact with the face, the elastic piece 4 undergoes expansion and contraction deformation under the force. When the elastic piece 4 is compressed, the resisting seat 110 can be resisted with the first step portion 24, so that the compression amount of the elastic piece 4 is not too large. When the elastic piece 4 stretches outward, the resisting seat 110 can be matched with the second step portion 41 to resist, so that the protection of the elastic piece 4 can be realized, and the service life of the elastic piece 4 is prolonged.

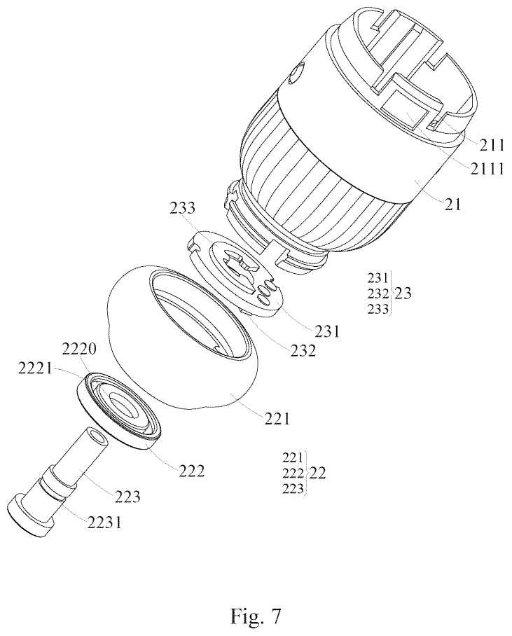

Referring to FIG. 7 and FIG. 11, the first end of the liquid discharge head body 2 is provided with an insert plate 211, and a slot 42 for insertion of the insert plate 211 is provided in the counter-position of the elastic piece 4. Specifically, the insert plate 211 may be arranged at the first end of the base 21 which is close to the elastic piece 4. This structure can realize the rapid positioning between the base 21 and the elastic piece 4 through the insert plate 211 and the slot 42, and also avoid the swing of the liquid discharge head body 2.

In an embodiment, the first end of the base 21 comprises two insert plates 211 and the elastic piece 4 comprises two slots 42 to position with the insert plate 211.

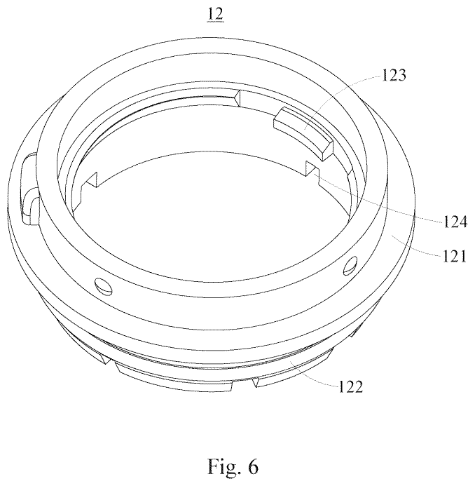

Referring to FIG. 6 and FIG. 7, the insert plate 211 has a limiting clamping groove 2111 and the housing 1 has a limiting protrusion 123 on the inner side wall. The limiting protrusion 123 inserts into the limiting clamping groove 2111. In a preferred embodiment, the limiting protrusion 123 is provided on the inner side wall of the bottom shell 12. This structure, through the cooperation of the limiting clamping groove 2111 and the limiting protrusion 123 prevents the loosening of the liquid discharge head body 2 and the housing 1 during the swing of the liquid discharge head body 2.

Referring to FIG. 3 and FIG. 11, the elastic piece 4 is provided with a refuge 43 for the first liquid discharge channel 10 to pass through. The inner side wall of the refuge 43 is convex inward with a boss 44. This structure can realize the avoidance of the first liquid discharge channel 10 through the refuge 43, so that a drain pipe 3 is connected with the first liquid discharge channel 10. The boss 44 can be used to resist the wire rod to avoid the elastic piece 4 in the process of expansion and contraction, and the wire rod is offset and disordered.

Referring to FIG. 4 and FIG. 5, the applicator further comprises a battery 6, a main circuit board 5 and a stimulation element (not shown). The battery 6 and the main circuit board 5 are installed in the housing 1. The stimulation element is installed on the main circuit board 5, and the battery 6 and the stimulation element are electrically connected with the main circuit board 5.

In an embodiment, the stimulation element may include but is not limited to LEDs, lasers, heating elements, cooling elements, vibration elements, electrodes, micro-current element or a combination thereof. The vibration element can be used to drive the housing to vibrate such that the liquid in the first liquid discharge channel 10, the drain pipe 3 and the second liquid discharge channel 20 can be vibrated and mixed, thereby helping to improve the coating effect. In addition, the stimulation element can drive the liquid discharge head body 2 to achieve alternate therapy to the face. The battery 6 is used to provide electric energy to drive the stimulation element.

Referring to FIG. 4, the main circuit board 5 and the battery 6 can be mounted on the bracket 13. The two ends of the bracket 13 are provided with a claw 131. The two claws 131 can clamp and fix the two ends of the main circuit board 5.

Referring to FIG. 4, a positioning rod 132 is installed on the bracket 13. The main circuit board 5 is provided with a positioning hole 50 for positioning the positioning rod 132 to insert. The cooperation of positioning rod 132 and the positioning hole 50, can improve the rapid positioning and installation of the main circuit board 5 and the bracket 13.

Referring to FIG. 4 and FIG. 5, two ends of the bracket 13 are provided with one or more positioning slots 133. The inner side wall of the shell 11 is provided with a positioning guide 117, and two ends of the positioning guide 117 can be inserted into two positioning slots 133, so that the battery 6 can be pressed on the bracket 13 to realize the installation and fixation of the battery 6.

Referring to FIG. 5 and FIG. 6, the top of the bottom shell 12 is provided with a limiting slot 124 for the positioning guide 117 to be inserted. Through the opposite insertion of positioning guide 117 and limiting slots 124, this structure can realize the fast positioning connection between the bottom shell 12 and the shell 11.

Referring to FIG. 5 and FIG. 7, the liquid discharge head body 2 comprises a base 21, a massage head 22 and a first plate 23 containing one or more stimulation elements. One end of base 21 is connected with elastic piece 4, and the other end of base 21 is connected with massage head 22. The first plate 23 is installed in the massage head 22, and the second liquid discharge channel 20 passes through the massage head 22. The first plate 23 is electrically connected with the battery 6. In an embodiment, the stimulation element is LEDs and the light emitted by the LEDs is emitted by the liquid outlet end of the massage head 22. In this structure, the light emitted by the LEDs on the first plate 23 is used to provide phototherapy on the skin of the face. By setting the liquid discharge head body 2 to the base 21 and the massage head 22, the maintenance of the first plate 23 can be realized through the disassembly and assembly between the base 21 and the massage head 22.

In an embodiment, one end of the base 21 close to the massage head 22 is a transparent part and made of PC (Polycarbonate, polycarbonate) material, so that the visualization of liquid flow in drain pipe 3 can be realized.

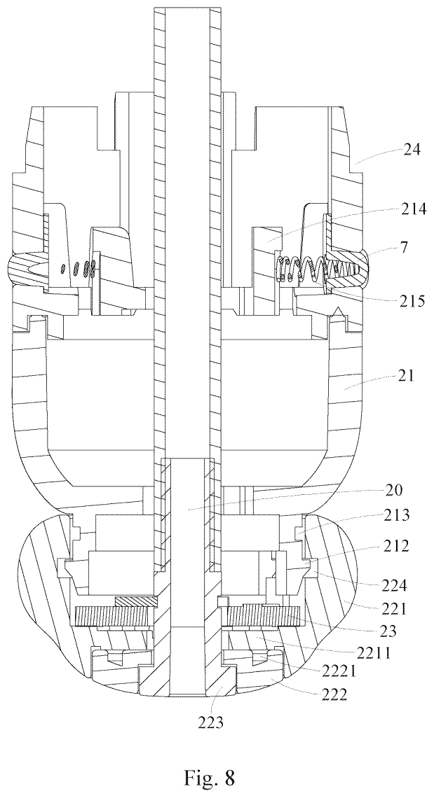

Referring to FIG. 8, one end of the base 21 close to the massage head 22 is provided with a clamp hook 212. The inner side wall of the massage head 22 is provided with a clamping groove 224. The clamp hook 212 fits into the clamping groove 224 to provide the detachable connection between the base 21 and the massage head 22.

Referring to FIG. 8, the outer side wall of one end of the base 21 close to the massage head 22 is provided with an annular mounting groove 213, and a scaling ring may be installed in the annular mounting groove 213. The gap between the base 21 and the massage head 22 can be filled by the sealing ring, so that stray light can be avoided from entering the inside of the massage head 22 and affecting the phototherapy effect of the first plate 23. The sealing ring also prevents impurities such as external dust from entering the inside of the massage head 22 and causing damage to the first plate 23. In an alternate embodiment, the inner side wall of the massage head 22 is provided with an annular groove, and a sealing ring may be installed in the annular groove. In an alternate embodiment, the outer side wall of the base 21 and the inner side wall of the massage head 22 are provided with annular grooves, and sealing rings are installed in each annular groove, so that the sealing performance between the base 21 and the massage head 22 can be improved.

Referring to FIG. 7 and FIG. 8, the massage head 22 comprises a bottom part 221, a lens 222 and a connecting guide rod 223. The bottom part 221 and the base 21 can be detachably connected with the clamping groove 224 using the clamp hook 212. The lens 222 is arranged at the liquid outlet end of the bottom part 221, and the connecting guide rod 223 is connected to connect the lens 222 and the bottom part 221, so that the lens 222 is fixed on the bottom part 221. The second liquid discharge channel 20 flows in the connecting guide rod 223, and the light emitted by the first plate 23 passes through the lens 222. In this structure, the light emitted by the first plate 23 can be emitted by the lens 222 after reflection, so that the phototherapy effect can be improved. The lens 222 can be connected with the bottom part 221 by connecting guide rod 223 to realize the disassembly of the massage head 22.

Referring to FIG. 7 to FIG. 9, the lens 222 is provided with a first through hole 2220 for the connecting guide rod 223 to pass through. The first plate 23 comprises a secondary circuit board 231, at least one stimulation element 232 and a locking piece 233. The secondary circuit board 231 has a second through hole 2310 for the connecting guide rod 223 to pass through. The at least one stimulation element 232 is installed on the side of the secondary circuit board 231 facing the lens 222, and the locking piece 233 is clamped on the connecting guide rod 223. The peripheral surface of the connecting guide rod 223 is provided with an annular guide groove 2231, and the locking piece 233 can be clamped in the annular guide groove 2231, so that the connection stability of the locking piece 233 and the connecting guide rod 223 can be improved. The connecting guide rod 223 detachably fixes the lens 222 and the first plate 23. The light emitted by the at least one stimulation element 232 falls perpendicularly onto the lens 222 and emits it.

Referring to FIG. 8 and FIG. 10, the inner side wall of the bottom part 221 extends inward to form a partition plate 2211. The first plate 23 and the lens 222 are arranged on two sides of the partition plate 2211. The partition plate 2211 is provided with a third through hole 2212 for the connecting guide rod 223 to pass through. A fourth through hole 2213 is further formed in the partition plate 2211, and the fourth through hole 2213 is arranged opposite to the stimulation element 232. In this structure, the lens 222 and the first plate 23 can be separated by the partition plate 2211 to avoid mutual interference. The light emitted by the stimulation element 232 can be irradiated to the lens 222 through the fourth through hole 2213 to avoid blocking.

Referring to FIG. 8 to FIG. 10, the first plate 23 comprises a plurality of LEDs arranged in a circular array. The number of the fourth through hole 2213 is the same as the number of LEDs. The plurality of fourth through hole 2213 is arranged in front of a plurality of LEDs. The side of the lens 222 facing the partition plate 2211 is provided with an annular groove portion 2221. The annular groove portion 2221 and a plurality of fourth through hole 2213 are arranged oppositely. Incorporating a plurality of LEDs, a plurality of fourth through hole 2213 and a lens 222, can improve the phototherapy intensity and phototherapy area and the phototherapy effect.

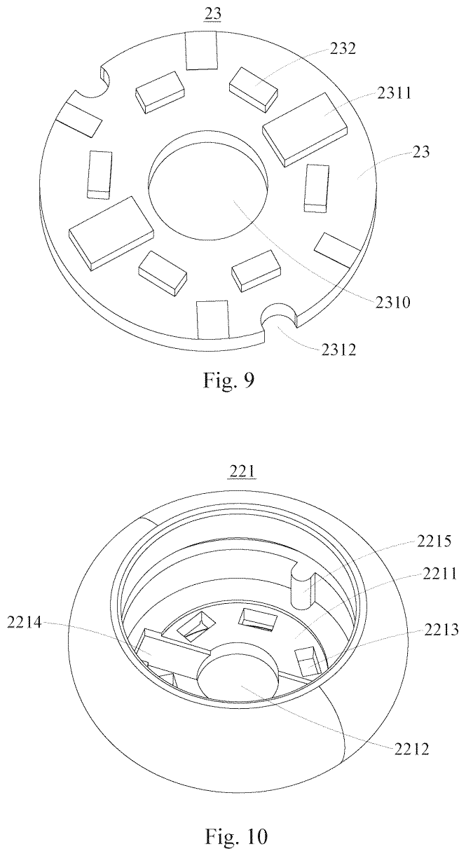

Referring to FIG. 9 and FIG. 10, a positioning groove 2214 is formed in the partition plate 2211, and a positioning piece 2311 inserted in the positioning groove 2214 is installed in the secondary circuit board 231. This structure, through the cooperation of the positioning piece 2311 and the positioning groove 2214, can improve the rapid positioning and installation of the secondary circuit board 231 and the partition plate 2211. The number of positioning groove 2214 and positioning pieces 2311 can be plurality, and more specifically are two in number, so that the positioning effect of the secondary circuit board 231 and the partition plate 2211 is improved. The positioning piece 2311 and the positioning groove 2214 are bonded and fixed, so that the first plate 23 can be fixedly installed.

Referring to FIG. 9 and FIG. 10, a positioning guide rod 2215 inserted into the avoidance notch 2312 is installed in the secondary circuit board 231 and a positioning guide rod 2215 inserted into the avoidance notch 2312 is installed on the inner side wall of the bottom part 221. The counter-alignment of the positioning guide rod 2215 and the avoidance notch 2312, can realize the rapid positioning and installation of the first plate 23 and avoid the position deviation of the first plate 23 in the process of swinging.

Referring to FIG. 7, the annular groove portion 2221 is roughly in a U-shaped structure, and the bottom surface of the annular groove portion 2221 is inclined to be arranged, that is, the bottom surface of the annular groove portion 2221 is inclined in the axis direction of the second liquid discharge channel 20. When the light emitted by the stimulation element 232 is perpendicular to the bottom surface of the annular groove portion 2221, the bottom surface of the annular groove portion 2221 reflect the light. The reflected light rays can be emitted into the lens 222 and the uniformity of the light reflected or refracted through the lens 222 is high, and then the light uniformity and phototherapy effect is improved.

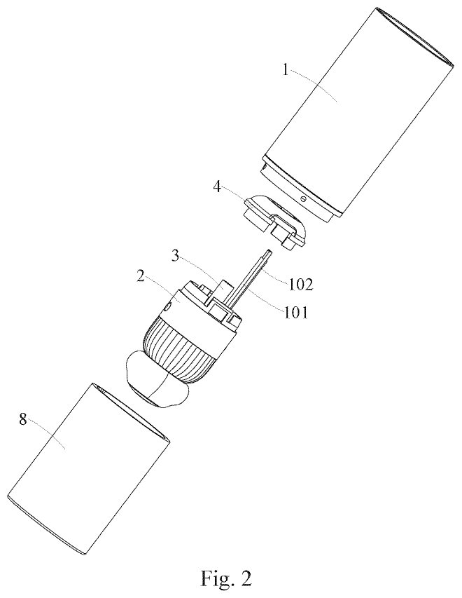

Referring to FIG. 2, the secondary circuit board 231 and the main circuit board 5 can be electrically connected through wires, and a hose 101 can be sleeved on the wires to realize the protection of the wires. The hose 101 is transparent and colored to visualize the flow of liquid from the hose 101.

Referring to FIG. 2, a luminescent filament 102 is installed on the main circuit board 5, and the luminescent filament 102 is electrically connected with the main circuit board 5. The luminescent filament 102 can emit color light after being energized, which can simulate the flexible effect of jellyfish antennae. The hose 101 and the luminescent filament 102 are supported by the boss 44. In a preferred embodiment, the luminescent filament 102 emits red color light. The light emitted by the luminescent filament 102 illuminate the hose 101.

Referring to FIG. 2 and FIG. 8, the applicator further comprises a conductive needle 7 and a charging unit 8. The conductive needle 7 is installed on the liquid discharge head body 2. The conductive needle 7 is electrically connected with the main circuit board 5, and the charging unit 8 is electrically connected with the conductive needle 7. The conductive needle can charge battery 6 by the charging unit 8.

Referring to FIG. 8, the conductive needle 7 is installed on the base 21. Specifically, a side plate 214 and a spring 215 is installed on the base 21, and an opening is provided in the base 21 for conductive needle 7 to stretch out. One end of the spring 215 is butted against the side plate 214, and the other end of the spring 215 is butted against the conductive needle 7. Through the spring 215, the resisting of the conductive needle 7 is realized, and the connection tightness of the conductive needle 7 and the charging unit 8 is ensured, and the electrical connection stability of the conductive needle 7 and the charging unit 8 is improved. In an embodiment, a pair of conductive needle is provided at the two ends of the base 21.

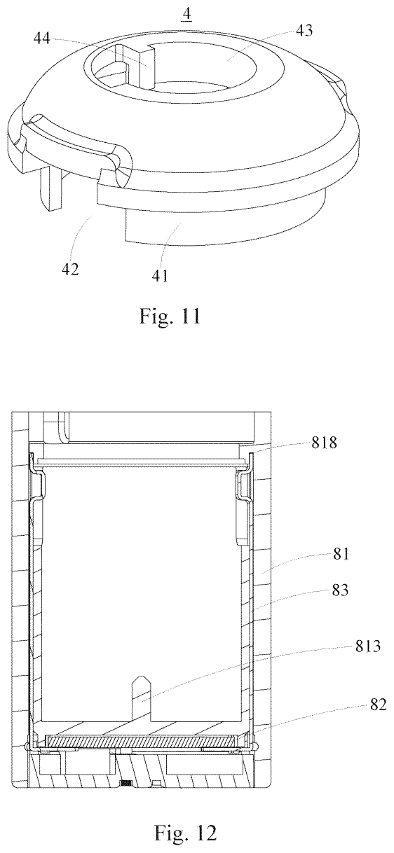

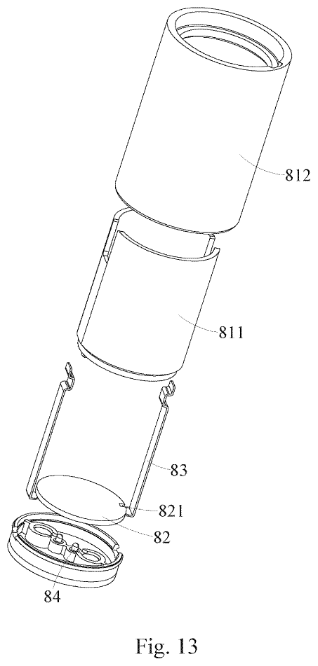

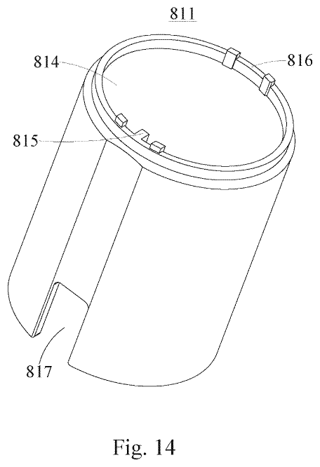

Referring to FIG. 12 and FIG. 13, the charging unit 8 comprises a cover body 81, a charging circuit board 82, a conductive piece 83 and a charging pin 84. The charging circuit board 82, the conductive piece 83 and the charging pin 84 are installed in the cover body 81. A first end of the conductive piece 83 is electrically connected with the charging circuit board 82, and a second end of the conductive piece 83 is butted against the conductive needle 7. A first end of the charging pin 84 is electrically connected with the charging circuit board 82, and a second end of the charging pin 84 extends out of the cover body 81. The cover body 81 can be hooded on the discharge head body 2 to realize the shielding of the second liquid discharge channel 20. When the cover body 81 is located on the liquid discharge head body 2, the conductive piece 83 can connect with the conductive needle 7 to provide electrical connection. When the charging pin 84 is connected to an external circuit, the battery 6 can be charged.

Referring to FIG. 12 and FIG. 13, the cover body 81 comprises an inner tube 811 and an outer tube 812 sleeved on the inner tube 811. The inner tube 811 is mounted and fixed on the outer tube 812, and the conductive piece 83 may be arranged in the gap between the inner tube 811 and the outer tube 812. The charging pin 84 can be installed on the outer tube 812. The inner tube 811 is provided with a sealing guide rod 813 inserted into the second liquid discharge channel 20 on the inner side wall facing the liquid discharge head body 2. The second liquid discharge channel 20 can be sealed by the sealing guide rod 813 to avoid leakage.

Referring to FIG. 13 and FIG. 14, an accommodating groove 814 for housing the charging circuit board 82 is provided in the outer side wall of the inner tube 811 deviating from the sealing guide rod 813, and a card holder 815 is arranged on the bottom surface of the accommodating groove 814. A positioning notch 821 is provided in the charging circuit board 82 for inserting into the accommodating groove 814, such that the charging circuit board 82 and the inner tube 811 can be installed and positioned. The inner side wall of the accommodating groove 814 is provided with a first avoidance gap 816 for the conductive piece 83 to extend into, so as to realize the avoidance of the conductive piece 83.

Referring to FIG. 12 and FIG. 14, the conductive piece 83 is bent to form a U-shaped structure that is countered with the conductive needle 7, and a second avoidance gap 817 is provided on the inner tube 811 at a position corresponding to the U-shaped structure to realize the butting contact between the conductive piece 83 and the conductive needle 7.

Referring to FIG. 12, the inner side wall of the outer tube 812 is provided with the limiting groove 818 for the free end of the conductive piece 83 to protrude. Through the limiting groove 818, the limiting fixation of the conductive piece 83 can be realized, the installation stability of the conductive piece 83 is improved, and the resisting tightness of the conductive piece 83 and the conductive needle 7 is improved.

Referring to FIG. 13, the inner tube 811 has two conductive piece 83 and two charging pins 84 located at two ends of the inner tube 811. Each of the two conductive piece are in contact with the conductive needle 7. The two conductive needles 7 can be used as a positive electrode and a negative electrode.

Referring to FIG. 2, FIG. 7 and FIG. 8, the top portion of the applicator comprises a massage head 22. The massage head 22 comprises a massage head 22, a first plate 23, a connecting guide rod 223 and a locking piece 233. The massage head 22 has a first through hole 2220 in the bottom part 221. The first plate 23 is arranged in the massage head 22. The first plate 23 has a second through hole 2310, the second through hole 2310 is arranged directly opposite the first through hole 2220. The first plate 23 is used for providing alternate therapy to the user. The connecting guide rod 223 passes through the first through hole 2220 and the second through hole 2310. One end of the connecting guide rod 223 is butted against the edge of the first through hole 2220, and the other end of connecting guide rod 223 can stretch into the massage head 22. The locking piece 233 is detachably connected with the connecting guide rod 223 at an end extending out of the second through hole 2310. When the locking piece 233 is installed on the connecting guide rod 223, the locking piece 233 can cooperate with the inner side wall of the massage head 22 to clamp and fix the first plate 23. When the locking piece 233 is installed on the connecting guide rod 223, the locking piece 233 clamps the first plate 23 with the inner side wall of the massage head 22, so as to realize the installation and fixation of the first plate 23. The massage head 22 assembly can be dismantled by unlocking the locking piece 233 from the connecting guide rod 223 and dismantling of the first plate 23 can be performed conveniently and quickly.

Referring to FIG. 7 and FIG. 8, the locking piece 233 comprises a snap ring. The peripheral surface of the connecting guide rod 223 is provided with an annular guide groove 2231, and the locking piece clamp is arranged in the annular guide groove 2231. Specifically, the annular guide groove 2231 may be arranged at one end of the connecting guide rod 223 extending out of the second through hole 2310. The locking piece 233 clamps and fit on the annular guide groove 2231 of the connecting guide rod 223, thereby improving the connection stability of the locking piece 233 and the connecting guide rod 223, and provides a convenient and a quick disassembly and assembly between the locking piece 233 and the connecting guide rod 223.

In an embodiment, the connecting guide rod 223 can be a bolt, and the locking piece can be a nut. Through the threaded connection of bolt and nut, the detachable connection between the locking piece and the connecting guide rod 223 can be realized. The nut and the inner side wall of the massage head 22 can be used to clamp and fix the first plate 23. In another embodiment, the connecting guide rod 223 is a magnetic suction piece made of ferrous material, the locking piece is a magnet, and a magnetic connection can be realized between the locking piece and the connecting guide rod 223.

Referring to FIG. 8 to FIG. 10, in a specific embodiment, the stimulation element in the massage head comprises one or more LEDs for providing phototherapy to a user. The first plate 23 is provided with the stimulation element 232 (luminous lamp beads). The partition plate 2211 is installed in the massage head body 2, and a first through hole 2220 is formed in the partition plate 2211, and a fourth through hole 2213 is formed in the position corresponding to the stimulation element 232 (luminous lamp bead) on the partition plate 2211. The massage head 22 further comprises the lens 222, the first plate 23 and the lens 222. The lens 222 is arranged on opposite side of the partition plate 2211 and the third through hole 2212 for the connecting guide rod 223 to pass through is formed in the lens 222. The first plate 23 comprises a secondary circuit board 231 and the stimulation element 232 (luminescent lamp bead) are mounted on the side of the secondary circuit board 231 facing the partition plate 2211. The stimulation element 232 (luminous lamp bead) is electrically connected with the secondary circuit board 231. The light can be processed through the lens 222, and the light effect can be improved. The fourth through hole 2213 is used to pass the light emitted by the stimulation element 232 (luminous lamp bead) and shines on the lens 222. The first plate 23 is separated from the lens 222 through the partition plate 2211, the connecting guide rod 223 passes through the third through hole 2220, and the lens 222 can be fixed on the partition plate 2211 using the connecting guide rod 223 and the locking piece 233.

Referring to FIG. 9 and FIG. 10, the secondary circuit board 231 is further provided with a positioning piece 2311. The positioning piece 2311 and the stimulation element 232 are arranged at interval. The partition plate 2211 is provided with the positioning groove 2214 to insert the positioning piece 2311. The cooperation of the positioning piece 2311 and the positioning groove 2214 improves the rapid positioning and installation of the secondary circuit board 231 and the partition plate 2211. In an embodiment, the massage head 22 comprises a plurality of the positioning groove 2214 and the positioning pieces 2311.

Referring to FIG. 9 and FIG. 10, the stimulation element 232 are a plurality of luminous lamp beads arranged in a circular array on the first plate 23. The fourth through hole 2213 are same in number as the number of luminous lamp beads. The plurality of fourth through hole 2213 is arranged opposite to a plurality of luminous lamp beads. This arrangement of the plurality of luminous lamp beads and the plurality of fourth through hole 2213 improves the phototherapy intensity and phototherapy area and thus improve the phototherapy effect.

Referring to FIG. 8, the annular groove portion 2221 is formed in the side of the lens 222 facing the partition plate 2211, and the annular groove portion 2221 is directly arranged opposite to the plurality of fourth through hole 2213. The light emitted by the plurality of luminous lamp beads can be emitted into the annular groove portion 2221. The annular groove portion 2221 can reflect the light that helps to improve the uniformity of light output, thereby improving the phototherapy effect.

The annular groove portion 2221 is roughly in a U-shaped structure, and the bottom surface of the annular groove portion 2221 is obliquely arranged, such that the bottom surface of the annular groove portion 2221 is inclined to the axis direction of the connecting guide rod 223. When the light emitted by the luminous lamp bead is perpendicular to the bottom surface of the annular groove portion 2221, the bottom surface of the annular groove portion 2221 reflects the light rays and the reflected light rays can be emitted into the lens 222 and the uniformity of the light reflected or refracted through the lens 222 is high. This helps to improve the uniformity of the light output and the phototherapy effect.

Referring to FIG. 9 and FIG. 10, the first plate 23 is provided with the avoidance notch 2312 arranged at intervals with the second through hole 2310. The positioning guide rod 2215 inserted into the avoidance notch 2312 is installed on the inner side wall of the massage head 22. The avoidance notch 2312 is arranged on the secondary circuit board 231. This prevents the position of the first plate 23 from shifting due to the swinging of the massage head 22 in process of massaging the face. In an embodiment, the avoidance notch 2312 and the positioning guide rods 2215 are plurality in numbers.

Referring to FIG. 7 and FIG. 10, the massage head 22 comprises the base 21 and the bottom part 221 detachably connected with the base 21. The bottom part 221 has the first through hole 2220, and the first plate 23 is arranged in the bottom part 221. This facilitates the disassembly and assembly of the first plate 23. The massage head 22 can be conveniently connected with the bottle or reservoir 100 through the base 21.

Referring to FIG. 8, one end of the base 21 close to the bottom part 221 is provided with a clamp hook 212, and the inner side wall of the bottom part 221 is provided with an clamping groove 224 for clamping with the clamp hook 212. This ensures the detachable connection between the base 21 and the bottom part 221.

Referring to FIG. 8, the outer side wall of one end of the base 21 close to the bottom part 221 is provided with an annular mounting groove 213, and a sealing ring may be installed in the annular mounting groove 213. The sealing ring fills the gap between the base 21 and the bottom part 221 to avoid stray light from entering the massage head 22 and avoid impurities such as external dust from entering the massage head 22 which can cause damage to the first plate 23.

Referring to FIG. 8, a liquid outlet hole is provided in the connecting guide rod 223. The liquid outlet hole is used for dispensing the liquid for the purpose of applying liquid on the face of the user.

The embodiment of the present application also provides a holding bottle. The holding bottle is a bottle or reservoir 100. The applicator with the massage head is installed at the liquid outlet end of the bottle or reservoir 100. Referring to FIGS. 1 to 3, the accommodating bottle may include the liquid discharge head body 2, the drain pipe 3 and the elastic piece 4. The liquid discharge head body 2 further comprises the housing 1 connected with the base 21. The main circuit board 5 and the battery 6 are installed in the housing 1. The first liquid discharge channel 10 is formed in housing 1, and two ends of housing 1 are liquid inlet end and liquid outlet end. The liquid inlet end and liquid outlet end of housing 1 are two ends of first liquid discharge channel 10.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. An applicator for applying a liquid, comprising:
a housing having a liquid inlet end configured to be connected to a liquid storage bottle;
a first liquid discharge channel in the housing;
a liquid discharge head body having a second liquid discharge channel;
a drain pipe to connect the first liquid discharge channel with the second liquid discharge channel; and
an elastic piece positioned between the housing and the liquid discharge head body, the elastic piece compress or decompress under an action of an external force.

2. The applicator of claim 1, wherein the elastic piece compresses when the liquid discharge head body swings around the housing, under the action of the external force and decompress when the external force is removed.

3. The applicator of claim 1, wherein the elastic piece allows 360 degree rotation of the liquid discharge head body and tilting the liquid discharge head body at an angle.

4. The applicator of claim 1, wherein an inner side wall of the housing extends inward to form a resisting seat, the resisting seat is positioned between a first step portion of the liquid discharge head body and a second step portion of the elastic piece.

5. The applicator of claim 1, wherein a first end of the liquid discharge head body has an insert plate that inserts into a slot provided on the elastic piece.

6. The applicator of claim 5, wherein the insert plate comprises a limiting clamping groove for fitting with a limiting protrusion provided on an inner side wall of the housing.

7. The applicator of claim 1, wherein the elastic piece is provided with a refuge to allow the first liquid discharge channel to pass through, and an inner side wall of the refuge is convex inward and is provided with a boss.

8. The applicator of claim 1, wherein the housing further comprises a bracket for supporting the elastic piece.

9. The applicator of claim 1 further comprising a conductive needle installed on the liquid discharge head body and a charging unit for charging a battery.

10. An applicator for applying a liquid, comprising:
a housing having a liquid inlet end configured to be connected to a liquid storage bottle;
a first liquid discharge channel in the housing;
a liquid discharge head body having a second liquid discharge channel connected to the first liquid discharge channel;
wherein the liquid discharge head body comprising:
a massage head body having a first through hole formed in centre of the massage head body;
a first plate having at least one stimulation element arranged in the massage head body, the first plate has a second through hole in centre in a position corresponding to the first through hole;
a connecting guide rod that passes through the first through hole and the second through hole; and
wherein the connecting guide rod has a liquid outlet hole.

11. The applicator of claim 10, wherein the applicator further comprises a locking piece to clamp the first plate with an inner side of the massage head body, the locking piece detachably connects with the connecting guide rod above the second through hole of the first plate.

12. The applicator of claim 11, wherein the connecting guide rod has an annular guide groove and the locking piece comprises a snap ring that snuggly fits into the annular guide groove on the connecting guide rod.

13. The applicator of claim 10, wherein the at least one stimulation element is selected from a group consisting of Light Emitting Diodes (LEDs), lasers, heating elements, cooling elements, vibration elements, electrodes, micro-current element and combinations thereof.

14. The applicator of claim 10, wherein the massage head comprises a partition plate and the first through hole is at the centre of the partition plate.

15. The applicator of claim 14, wherein the partition plate comprises a fourth through hole corresponding to the position of the at least one stimulation element on the first plate.

16. The applicator of claim 14, wherein the massage head further comprises a lens positioned above the partition plate, the lens has a third through hole for the connecting guide rod to pass through.

17. The applicator of claim 14, wherein the partition plate comprise one or more positioning grooves and the first plate comprise one or more positioning piece to fit into the one or more positioning grooves on the first plate.

18. The applicator of claim 10, wherein the first plate comprises one or more avoidance notch arranged at intervals with the second through hole, and an inner side wall of the massage head body is provided with one or more positioning guide rod for inserting into the one or more avoidance notch.

19. The applicator of claim 10 further comprising a top portion connected to the massage head body, the top portion has a housing for accommodating a main circuit board and a battery.

20. An applicator for applying a liquid, comprising:
a housing having a liquid inlet end configured to be connected to a liquid storage bottle;
a first liquid discharge channel in the housing;
a liquid discharge head body having a second liquid discharge channel;
a drain pipe to connect the first liquid discharge channel with the second liquid discharge channel; and
wherein the liquid discharge head body is transparent and includes a luminescent filament in a hose that emits a color light.
